(19) United States
(12) Patent Application Publication
(10) Pub. No.: US 2011/0190807 A1
(43) Pub. Date: Aug. 4, 2011

(54) REMOTE ISCHEMIC CONDITIONING FOR TREATMENT AND PREVENTION OF RESTENOSIS

(75) Inventors: ANDREW REDINGTON, Toronto (CA); CHRISTOPHER CALDARONE, Toronto (CA)

(73) Assignee: The Hospital for Sick Children, Toronto (CA)

(21) Appl. No.: 13/018,664

(22) Filed: Feb. 1, 2011

Related U.S. Application Data
(60) Provisional application No. 61/300,316, filed on Feb. 1, 2010.

(51) Int. Cl. A61B 17/12

(52) U.S. Cl. 606/201

(57) ABSTRACT

The invention provides methods for reducing the incidence and/or severity of restenosis through the use of remote ischemic conditioning.

### RELATED APPLICATIONS

This application claims the benefit of U.S. Provisional Application Ser. No. 61/300,316, filed on Feb. 1, 2010, entitled “TREATMENT AND PREVENTION OF RESTENOSIS”, the entire contents of which are incorporated by reference herein.

### BACKGROUND OF THE INVENTION

Restenosis, or renarrowing of a vessel or other narrowed biologic structure, is a common complication following dilatation or stent placement. It can occur in anywhere from 10-50% of patients. Certain drug-eluting stents are reportedly associated with a lower occurrence of restenosis. However these stents are also complicated by restenosis, and have their own drawbacks, not the least of which is cost. Patients in whom restenosis occurs typically must undergo a repeated procedure in order to re-expand or bypass the narrowing.

### SUMMARY OF THE INVENTION

The invention relates generally to the use of remote ischemic conditioning (RIC) to reduce the occurrence and severity of restenosis. Restenosis may occur following a medical procedure (or intervention) aimed at opening or widening a blood vessel or biologic tube (including but not restricted to esophagus, biliary tree, bronchus, and the like). Such procedures include but are not limited to stent placements and balloon angioplasty, both of which can cause vessel damage.

The invention therefore also contemplates the use of RIC on a subject that has or is likely to experience vessel damage that can lead to restenosis. In these subjects, RIC may be performed before and after the occurrence of an event, such as a medical procedure, that is likely to induce vessel damage. Alternatively, RIC may be performed before (pre-conditioning), during (per-conditioning), and/or after (post-conditioning) the occurrence of an event that is likely to induce vessel damage, in any combination or pre-, per- and post-conditioning.

In most instances, the invention contemplates that the subject will undergo more than one RIC regimen. For example, RIC may be performed multiple times in a single day and/or one or more times on multiple days. In other words, instead of performing a single RIC regimen prior to an event, the invention envisions performing multiple RIC regimens and such RIC regimens may occur in one day (e.g., before or after the event) or on more than one day (e.g., before and/or after the event).

The invention therefore provides, in one aspect, a method for reducing restenosis in a subject comprising performing a repeated remote ischemic conditioning (RIC) regimen on a subject having or at risk of developing restenosis. Reducing restenosis may comprise reducing the incidence of restenosis compared to a control subject or population, in one embodiment. Reducing restenosis may comprise reducing the severity of restenosis in a subject, in one embodiment. Reducing restenosis may comprise delaying the onset of restenosis in a subject (e.g., as compared to a control population), in one embodiment. The delay may be months or years in length, in some embodiments.

In one embodiment, restenosis occurs following a medical intervention.

In various embodiments, the repeated RIC regimen comprises more than one RIC regimen performed on a single day. In some embodiments, the repeated RIC regimen comprises two, three, four or five RIC regimens performed on a single day.

In various embodiments, the repeated RIC regimen comprises one or more RIC regimens on more than one day (e.g., one RIC regimen per day for a number of days, or more than one RIC regimen per day for more than one day). In some embodiments, the repeated RIC regimen comprises one or more RIC regimens performed on a daily basis for one month or longer. In some embodiments, the repeated RIC regimen comprises one or more RIC regimens performed intermittently for one month or longer.

In various embodiments, the repeated RIC regimen comprises more than one RIC regimen on more than one day.

In important embodiments, the subject is human.

In one embodiment, the subject will receive a medical intervention. In one embodiment, the repeated RIC regimen is performed before the medical intervention. In one embodiment, the repeated RIC regimen is performed after the medical intervention. In one embodiment, the repeated RIC regimen is performed before and after the medical intervention. In still other embodiments, RIC regimens are performed before and during the medical intervention, before, during and after the medical intervention, or during and after the medical intervention.

In one embodiment, the medical intervention is a stent placement or insertion (e.g., into a narrowing in the body). In one embodiment, the medical intervention is an intravascular stent placement into the narrowing. In one embodiment, the intravascular stent placement is an arterial stent placement. In one embodiment, the intravascular stent placement is a venous stent placement. In one embodiment, the intravascular stent placement is a bare-metal stent placement. In one embodiment, the intravascular stent placement is a drug-eluting stent placement.

In one embodiment, the medical intervention is angioplasty such as balloon angioplasty (e.g., used to expand a narrowing in the body).

In one embodiment, the medical intervention is a non-vascular stent placement. In one embodiment, the medical intervention is a esophageal stent placement, a tracheal stent placement, a urethral stent placement, or a bile duct stent placement.

In one embodiment, at least one RIC regimen (within the repeated RIC regimen) is performed within 24 hours of the medical intervention. In one embodiment, at least one RIC regimen is performed within 2 hours of the medical intervention. In one embodiment, at least one RIC regimen is performed within 1 hour of the medical intervention. Such RICs may be performed before and/or after the medical intervention.

Each RIC regimen may comprise two, three, four, five or more cycles of supra-systolic pressure followed by reperfusion. Each period of supra-systolic pressure may have a duration of about 30 seconds, about 1 minute, about 2 minutes, about 3 minutes, about 4 minutes, about 5 minutes, or longer. Each period of reperfusion may have a duration of about 30 seconds, about 1 minute, about 2 minutes, about 3 minutes, about 4 minutes, about 5 minutes, or longer. The duration of the supra-systolic pressure period may be the same as or different from the duration of the reperfusion period.

In one embodiment, at least one RIC regimen (within the repeated RIC regimen) comprises at least four cycles, each cycle comprising supra-systolic pressure and reperfusion. In one embodiment, at least one RIC regimen comprises more than one cycle comprising 5 minutes of supra-systolic pressure and 5 minutes of reperfusion.

The supra-systolic pressure may be 5, 10, 15, 20, 25, 30, 35 or more mm Hg above systolic pressure. In one embodiment, the supra-systolic pressure is a pressure that is at least 15 mmHg above systolic pressure. In other embodiments, the supra-systolic pressure may be 160, 170, 180, 190, 200, 210, 220, 230, 240, 250 or more mm Hg. In still other embodiments, the supra-systolic pressure may be expressed as a percentage of systolic pressure, including 101%, 102%, 103%, 104%, 105%, 106%, 107%, 108%, 109%, 110%, or more of systolic pressure.

In one embodiment, the repeated RIC regimen is performed at the same site. In one embodiment, the repeated RIC regimen is performed on a limb (e.g., an upper limb or a lower limb). In one embodiment, an individual RIC regimen or a repeated RIC regimen is performed using two or more devices such as two or more cuffs, optionally positioned at different sites on the body (e.g., one cuff per arm, or one cuff per leg, or one cuff on an arm and one cuff on a leg, etc.). Each cuff, whether one or more are used, may comprise a single bladder or multiple bladders, including two, three or more bladders.

In one embodiment, the method further comprises administering to the subject an anti-platelet agent. In one embodiment, the method further comprises administering to the subject an anti-inflammatory agent.

In one embodiment, the subject is administered aspirin. In one embodiment, the subject is administered an anti-platelet agent such as clopidogrel. In one embodiment, the subject is administered an anti-coagulant agent such as heparin. In one embodiment, the subject is administered a glycoprotein IIb/IIIa inhibitor such as eptifibatide or tirofiban. In one embodiment, the subject is administered a statin.

The invention further provides in yet other embodiments, kits comprising devices or device components for performing remote ischemic conditioning and stents or catheters. The device components may be cuffs, such as disposable cuffs, or liners or sleeves for such cuffs, preferably wherein such liners or sleeves are disposable. The kits may comprise one, two, three, four or more cuffs, liners or sleeves, and one or more stents or catheters.

In various embodiments, the subject may be administered two or more of these aforementioned agents.

These and other aspects and embodiments of the invention will be discussed in greater detail herein.

### BRIEF DESCRIPTION OF THE FIGURES

The accompanying drawings are not intended to be drawn to scale. In the figures, each identical or nearly identical component that is illustrated in various figures is represented by a like numeral. For purposes of clarity, not every component may be labeled in every drawing.

Various embodiments of the invention will now be described, by way of example, with reference to the accompanying figures, in which:

FIG. 1 is a schematic representation of one embodiment of a remote ischemic conditioning system, including a pneumatically inflatable cuff configured to contract about the limb of a subject.

FIG. 2 is a block diagram of one embodiment of an operating scheme of the RIC system.

FIG. 3 shows an alternate embodiment of a cuff configured to contract about the limb of a subject.

FIG. 4 shows the effect of remote conditioning performed before and for 7 days after a vascular injury in an iliac artery balloon injury model. All available individual measurements were used to create subject-weighted linear regression models for each parameter using maximum likelihood methodology for parameter estimation. Reported in addition to p-values are group means and standard error.

### DETAILED DESCRIPTION OF THE INVENTION

The invention relates to the finding that incidence and/or severity of restenosis can be reduced by deliberately and repeatedly performing cycles of induced transient ischemia and reperfusion in subjects. These subjects include those that are experiencing restenosis and those at risk of developing restenosis. In particular, these subjects include those that have undergone a medical procedure that is associated with restenosis. Thus, even subjects that do not manifest any symptoms of restenosis may be treated according to the invention, particularly for the purpose of delaying the onset, slowing (e.g., reducing the severity of) or completely preventing restenosis.

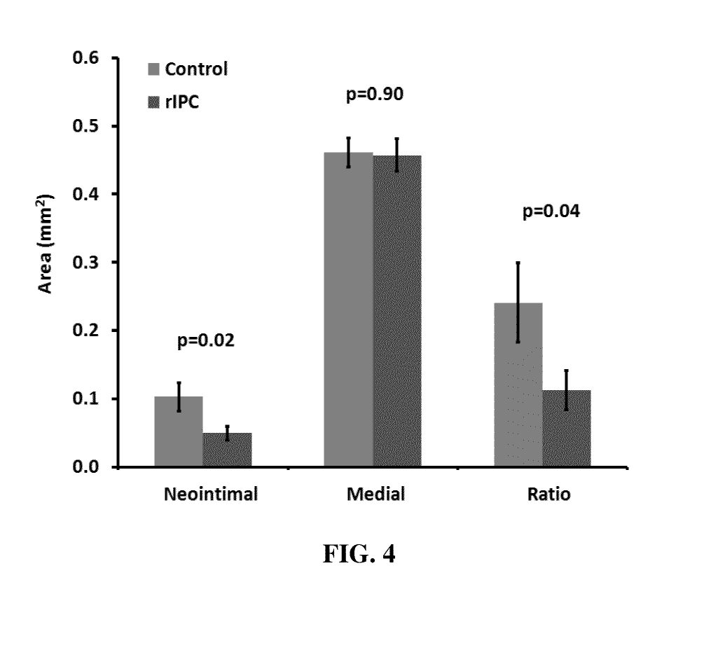

The invention contemplates, in some aspects, performing a repeated RIC regimen on a subject. As used herein, an RIC regimen (or an individual RIC regimen, as the terms are used interchangeably herein) means at least one cycle of an induced transient ischemic event or period (also referred to herein as a period of supra-systolic pressure) followed by a reperfusion event or period. An individual RIC regimen therefore may be comprised of 1, 2, 3, 4, 5, or more such cycles. RIC may be referred to as RIPC also, for example as indicated in FIG. 4.

Also as used herein, a repeated RIC regimen is two or more individual RIC regimens that occur on a single day and/or one or more RIC regimens that occur on a number of days. For example, the repeated RIC regimen may comprise performing multiple RIC regimens on a single day, or performing single RIC regimens on a number of days, or performing multiple RIC regimens on a number of days. If the repeated RIC regimen occurs on a single day, the time between individual regimens may be at least 10 minutes, at least 20 minutes, at least 40 minutes, at least 1 hour, at least 2 hours, or at least 6 hours, for example.

As should be clear, there is no requirement that any or all of the RIC regimens in a repeated RIC regimen be identical with respect to timing, number of cycles per regimen, supra-systolic pressure, location, and the like.

RIC is typically performed in an area of the body that is remote to the area that is receiving the medical intervention. Typically, RIC is performed on a limb such as an upper or lower limb. The repeated RIC regimen may be performed on a single site or on multiple sites in the body. For example, the repeated RIC regimen may comprise a first RIC regimen performed on the right upper arm, followed by a second RIC regimen performed on the left upper arm. The repeated RIC regimen may comprise alternation between sites on the body. In some instances, an RIC regimen may be performed on a subject at two different sites at overlapping times including simultaneously. In such instances, two devices may be used, as described below.

The subjects of the invention will preferably be humans, although non-human subjects are also contemplated. Essentially, any subject that can experience restenosis can be treated according to the invention. In some instances, the subjects are not at risk of myocardial infarction.

### Medical Procedures/Interventions

Medical interventions according to the invention include interventions that are performed to expand an abnormal narrowing in a subject and/or those that induce or are likely to induce vessel damage in a subject. The subjects to be treated according to the invention include those who have experienced (or are experiencing) a narrowing in a vessel. The subjects to be treated according to the invention include those who have undergone a medical intervention that induced or is likely to induce vessel damage. The subjects also include those who are scheduled to undergo such a medical intervention. These interventions may be elective or emergency procedures. These interventions therefore are associated with restenosis. In some instances, these interventions do not themselves produce an ischemic environment in the subject.

Medical interventions that are known to induce or are likely to induce vessel damage may be any surgical or non-surgical procedure that results in damage to any vessel in the body. The vessel may be a blood vessel such as an artery or a vein. The vessel may be a non-blood vessel (e.g., a vessel that carries a fluid other than, or in addition to, blood) such as the bile duct, the esophagus, the intestine (including large and small intestine), the trachea, the urethra, the Eustachian tube, and the like.

An example of such an intervention is a stent placement (or insertion or implantation). Stent placement or insertion may occur in any vessel of the body including many of the vessels discussed herein, and in any region of the body (e.g., in the brain, such as an intracranial stent), preferably provided that the RIC regimen is performed remotely to the location of the stent. Commonly, stent placement occurs intravascularly in an artery or in a vein. Stent placement may also occur in other vessels including in the bile duct, in the esophagus, in the Eustachian tube, and in the trachea. Stent placement may be used in any vessel to correct or ameliorate a narrowing of the vessel.

The stents may be of any type, including “bare” stents (such as bare-metal stents, used as vascular stents) and drug-eluting stents. Drug-eluting stents, as used herein, refer to stents which are coated with or otherwise comprise one or more therapeutic agents. Bare stents, on the other hand, do not comprise such agents. Bare and drug-eluting stents are known in the art.

Another example of a medical intervention is angioplasty (or percutaneous transluminal coronary angioplasty (PTCA)). Restenosis has been reported to occur in 30-50% of subjects who have undergone simple balloon angioplasty.

Those of ordinary skill in the art are readily aware of other medical interventions that cause vessel damage and/or that are associated with restenosis. It is to be understood that the invention intends to embrace the treatment of subjects undergoing any such intervention.

### Repeated RIC and Timing

The repeated RIC regimen may be performed before and/or during and/or after the medical intervention (e.g., before; before and during; before and after; before, during and after; during; during and after; or after the medical intervention or other event likely to induce vessel damage).

In some embodiments, the repeated RIC regimen is performed, in whole or in part, before the medical intervention. In such instances, at least one RIC regimen may be performed within 48 hours, within 24 hours, within 12 hours, within 6 hours, within 4 hours, within 2 hours, within 1 hour, within 30 minutes, within 20 minutes, within 10 minutes, within 5 minutes, or just immediately prior to the medical intervention.

In some embodiments, the repeated RIC regimen is performed, in whole or in part, after the medical intervention. In such instances, at least one RIC regimen may be performed within 48 hours, within 24 hours, within 12 hours, within 6 hours, within 4 hours, within 2 hours, within 1 hour, within 30 minutes, within 20 minutes, within 10 minutes, within 5 minutes, or just immediately after the medical intervention.

In some embodiments, the repeated RIC regimen spans a number of days, including 2, 3, 4, 5, 6, 7, 8, 9, 10, 15, or 30 or more days, or 1, 2, 3, 4, 5, 6 or more months. It is to be understood that in such instances, a subject may undergo an RIC regimen daily, every 2, 3, 4, 5, or 6 days, every week, every 2, 3, 4 weeks, every month, every 2, 3, 4, 5, 6 months, for example. Additionally, the RIC regimens may be performed in a non-regular, or random, manner.

### Reducing Restenosis

As used herein, restenosis refers to a renarrowing of a vessel (or other structure) after a procedure performed to relieve a narrowing. The invention aims, in some instances, to reduce the occurrence (or incidence) of restenosis in a subject, and/or to reduce the severity or degree of the restenosis, and/or to reduce or ameliorate the symptoms associated with restenosis.

A reduced occurrence of restenosis can be determined by comparing the treated subject to another subject, or more preferably a population of subjects, that has not received the repeated RIC regimen but is otherwise medically comparable to the treated subject. The average time of restenosis in this control group is compared to that of the treated subject, and a delayed onset of restenosis in the treated subject relative to the control is indicative of a reduced occurrence.

A reduction in the severity or degree of restenosis may be measured directly or indirectly. For example, the severity or degree of restenosis may be measured directly through, for example, measurement of a vessel diameter. Indirect measurements may include functional measurements. The nature of the functional measurement will depend upon the nature and normal function of the damaged vessel. An example of a functional measurement is flow rate and flow quality through the vessel. These measurements are preferably made when the restenosis is likely to occur, based on historical data from comparable but untreated subjects. Such timing may be days, weeks, months or years following treatment.

Analysis of symptoms relating to restenosis will also depend on the nature of the vessel(s) that may restenose. If restenosis may occur in the vasculature, then symptoms include any cardiovascular symptoms relating to blood flow impairment, including but not limited to cardiac and cerebral symptoms. These may include chest pain (angina), particularly following physical exertion, unusual fatigue, shortness of breath, and chest pressure.

Biological markers may also be measured as an indicator of restenosis. An example of a biological marker is troponin, which is elevated in the presence of restenosis.

Various tests are available to detect restenosis including imaging tests (e.g., CT, magnetic resonance imaging, radionuclide imaging, angiography, Doppler ultrasound, MRA, etc.), and functional tests such as an exercise stress test.

### Additional Therapies

The repeated RIC regimen of the invention may be used in combination with other therapies or procedures aimed at reducing restenosis. These therapies include local intravascular radiation (brachytherapy) and various chemotherapies such as inhibitors of platelet function, agents that reduce platelet count, anti-coagulant agents, fibrinolytic agents, anti-inflammatory agents, lipid reducing agents, direct thrombin inhibitors, glycoprotein IIb/IIIa receptor inhibitors, agents that bind to cellular adhesion molecules and inhibit the ability of white blood cells to attach to such molecules, calcium channel blockers, beta-adrenergic receptor blockers, cyclooxygenase-2 inhibitors, and angiotensin system inhibitors. Depending upon the embodiment, one or more of these agents may be administered before, simultaneously with or following the repeated RIC regimen and/or before, simultaneously with or following the medical intervention.

Fibrinolytic agents are agents that lyse a thrombus (e.g., a blood clot), usually through the dissolution of fibrin by enzymatic action. Examples include but are not limited to ancrod, anistreplase, bisobrin lactate, brinolase, Hageman factor (i.e. factor XII) fragments, molsidomine, plasminogen activators such as streptokinase, tissue plasminogen activators (TPA) and urokinase, and plasmin and plasminogen.

Anti-coagulant agents are agents that inhibit the coagulation pathway by impacting negatively upon the production, deposition, cleavage and/or activation of factors essential in the formation of a blood clot. Anti-coagulant agents include but are not limited to vitamin K antagonists such as coumarin and coumarin derivatives (e.g., warfarin sodium); glycosoaminoglycans such as heparins both in unfractionated form and in low molecular weight form; ardeparin sodium, bivalirudin, bromindione, coumarin dalteparin sodium, desirudin, dicumarol, lyapolate sodium, nafamostat mesylate, phenprocoumon, sulfatide, tinzaparin sodium, inhibitors of factor Xa, factor TFPI, factor VIIa, factor IXc, factor Va, factor VIIIa as well as inhibitors of other coagulation factors.

Inhibitors of platelet function are agents that impair the ability of mature platelets to perform their normal physiological roles (i.e., their normal function). Examples include but are not limited to acadesine, anagrelide, anipamil, argatroban, aspirin, clopidogrel, cyclooxygenase inhibitors such as nonsteroidal anti-inflammatory drugs and the synthetic compound FR-122047, danaparoid sodium, dazoxiben hydrochloride, diadenosine 5′,5′″-P1,P4-tetraphosphate (Ap4A) analogs, difibrotide, dilazep dihydrochloride, 1,2- and 1,3-glyceryl dinitrate, dipyridamole, dopamine and 3-methoxytyramine, efegatran sulfate, enoxaparin sodium, glucagon, glycoprotein IIb/IIIa antagonists such as Ro-43-8857 and L-700,462, ifetroban, ifetroban sodium, iloprost, isocarbacyclin methyl ester, isosorbide-5-mononitrate, itazigrel, ketanserin and BM-13.177, lamifiban, lifarizine, molsidomine, nifedipine, oxagrelate, PGE, platelet activating factor antagonists such as lexipafant, prostacyclin (PGI₂), pyrazines, pyridinol carbamate, ReoPro (i.e., abciximab), sulfinpyrazone, synthetic compounds BN-50727, BN-52021, CV-4151, E-5510, FK-409, GU-7, KB-2796, KBT-3022, KC-404, KF-4939, OP-41483, TRK-100, TA-3090, TFC-612 and ZK-36374, 2,4,5,7-tetrathiaoctane, 2,4,5,7-tetrathiaoctane 2,2-dioxide, 2,4,5-trithiahexane, theophyllin pentoxifyllin, thromboxane and thromboxane synthetase inhibitors such as picotamide and sulotroban, ticlopidine, tirofiban, trapidil and ticlopidine, trifenagrel, trilinolein, 3-substituted 5,6-bis(4-methoxyphenyl)-1,2,4-triazines, and antibodies to glycoprotein IIb/IIIa as well as those disclosed in U.S. Pat. No. 5,440,020, and anti-serotonin drugs, Clopridogrel; Sulfinpyrazone; Aspirin; Dipyridamole; Clofibrate; Pyridinol Carbamate; PGE; Glucagon; Antiserotonin drugs; Caffeine; Theophyllin Pentoxifyllin; Ticlopidine.

Anti-inflammatory agents include Alclofenac; Alclometasone Dipropionate; Algestone Acetonide; Alpha Amylase; Amcinafal; Amcinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lornoxicam; Loteprednol Etabonate; Meclofenamate Sodium; Meclofenamic Acid; Meclorisone Dibutyrate; Mefenamic Acid; Mesalamine; Meseclazone; Methylprednisolone Suleptanate; Morniflumate; Nabumetone; Naproxen; Naproxen Sodium; Naproxol; Nimazone; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit; Salcolex; Salnacedin; Salsalate; Salycilates; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetrydamine; Tiopinac; Tixocortol Pivalate; Tolmetin; Tolmetin Sodium; Triclonide; Triflumidate; Zidometacin; Glucocorticoids; Zomepirac Sodium. One preferred anti-inflammatory agent is aspirin.

Lipid reducing agents include gemfibrozil, cholystyramine, colestipol, nicotinic acid, probucol lovastatin, fluvastatin, simvastatin, atorvastatin, pravastatin, cirivastatin.

Direct thrombin inhibitors include hirudin, hirugen, hirulog, agatroban, PPACK, thrombin aptamers.

Glycoprotein IIb/IIIa receptor inhibitors are both antibodies and non-antibodies, and include but are not limited to ReoPro (abcixamab), lamifiban, tirofiban.

Calcium channel blockers are a chemically diverse class of compounds having important therapeutic value in the control of a variety of diseases including several cardiovascular disorders, such as hypertension, angina, and cardiac arrhythmias (Fleckenstein, Cir. Res. v. 52, (suppl. 1), p. 13-16 (1983); Fleckenstein, Experimental Facts and Therapeutic Prospects, John Wiley, New York (1983); McCall, D., Curr Pract Cardiol, v. 10, p. 1-11 (1985)). Calcium channel blockers are a heterogeneous group of drugs that prevent or slow the entry of calcium into cells by regulating cellular calcium channels. (Remington, The Science and Practice of Pharmacy, Nineteenth Edition, Mack Publishing Company, Easton, Pa., p. 963 (1995)). Most of the currently available calcium channel blockers, and useful according to the present invention, belong to one of three major chemical groups of drugs, the dihydropyridines, such as nifedipine, the phenyl alkyl amines, such as verapamil, and the benzothiazepines, such as diltiazem. Other calcium channel blockers useful according to the invention, include, but are not limited to, amrinone, amlodipine, bencyclane, felodipine, fendiline, flunarizine, isradipine, nicardipine, nimodipine, perhexylene, gallopamil, tiapamil and tiapamil analogues (such as 1993RO-11-2933), phenyloin, barbiturates, and the peptides dynorphin, omega-conotoxin, and omega-agatoxin, and the like and/or pharmaceutically acceptable salts thereof.

Beta-adrenergic receptor blocking agents are a class of drugs that antagonize the cardiovascular effects of catecholamines in angina pectoris, hypertension, and cardiac arrhythmias. Beta-adrenergic receptor blockers include, but are not limited to, atenolol, acebutolol, alprenolol, befunolol, betaxolol, bunitrolol, carteolol, celiprolol, hydroxalol, indenolol, labetalol, levobunolol, mepindolol, methypranol, metindol, metoprolol, metrizoranolol, oxprenolol, pindolol, propranolol, practolol, practolol, sotalolnadolol, tiprenolol, tomalolol, timolol, bupranolol, penbutolol, trimepranol, 2-(3-(1,1-dimethylethyl)-amino-2-hydroxypropoxy)-3-pyridenecarbonitrilHCl, 1-butylamino-3-(2,5-dichlorophenoxy)-2-propanol, 1-isopropylamino-3-(4-(2-cyclopropylmethoxyethyl)phenoxy)-2-propanol, 3-isopropylamino-1-(7-methylindan-4-yloxy)-2-butanol, 2-(3-t-butylamino-2-hydroxy-propylthio)-4-(5-carbamoyl-2-thienyl)thiazol, 7-(2-hydroxy-3-t-butylaminpropoxy)phthalide. The above-identified compounds can be used as isomeric mixtures, or in their respective levorotating or dextrorotating form.

A number of selective “COX-2 inhibitors” are known in the art. These include, but are not limited to, COX-2 inhibitors described in U.S. Pat. No. 5,474,995 “Phenyl heterocycles as cox-2 inhibitors”; U.S. Pat. No. 5,521,213 “Diaryl bicyclic heterocycles as inhibitors of cyclooxygenase-2”; U.S. Pat. No. 5,536,752 “Phenyl heterocycles as COX-2 inhibitors”; U.S. Pat. No. 5,550,142 “Phenyl heterocycles as COX-2 inhibitors”; U.S. Pat. No. 5,552,422 “Aryl substituted 5,5 fused aromatic nitrogen compounds as anti-inflammatory agents”; U.S. Pat. No. 5,604,253 “N-benzylindol-3-yl propanoic acid derivatives as cyclooxygenase inhibitors”; U.S. Pat. No. 5,604,260 “5-methanesulfonamido-1-indanones as an inhibitor of cyclooxygenase-2”; U.S. Pat. No. 5,639,780 N-benzyl indol-3-yl butanoic acid derivatives as cyclooxygenase inhibitors”; U.S. Pat. No. 5,677,318 Diphenyl-1,2-3-thiadiazoles as anti-inflammatory agents”; U.S. Pat. No. 5,691,374 “Diaryl-5-oxygenated-2-(5H)-furanones as COX-2 inhibitors”; U.S. Pat. No. 5,698,584 “3,4-diaryl-2-hydroxy-2,5-dihydrofurans as prodrugs to COX-2 inhibitors”; U.S. Pat. No. 5,710,140 “Phenyl heterocycles as COX-2 inhibitors”; U.S. Pat. No. 5,733,909 “Diphenyl stilbenes as prodrugs to COX-2 inhibitors”; U.S. Pat. No. 5,789,413 “Alkylated styrenes as prodrugs to COX-2 inhibitors”; U.S. Pat. No. 5,817,700 “Bisaryl cyclobutenes derivatives as cyclooxygenase inhibitors”; U.S. Pat. No. 5,849,943 “Stilbene derivatives useful as cyclooxygenase-2 inhibitors”; U.S. Pat. No. 5,861,419 “Substituted pyridines as selective cyclooxygenase-2 inhibitors”; U.S. Pat. No. 5,922,742 “Pyridinyl-2-cyclopenten-1-ones as selective cyclooxygenase-2 inhibitors”; U.S. Pat. No. 5,925,631 “Alkylated styrenes as prodrugs to COX-2 inhibitors”; all of which are commonly assigned to Merck Frosst Canada, Inc. (Kirkland, Calif.). Additional COX-2 inhibitors are also described in U.S. Pat. No. 5,643,933, assigned to G. D. Searle & Co. (Skokie, Ill.), entitled: “Substituted sulfonylphenylheterocycles as cyclooxygenase-2 and 5-lipoxygenase inhibitors.”

A number of the above-identified COX-2 inhibitors are prodrugs of selective COX-2 inhibitors, and exert their action by conversion in vivo to the active and selective COX-2 inhibitors. The active and selective COX-2 inhibitors formed from the above-identified COX-2 inhibitor prodrugs are described in detail in WO 95/00501, published Jan. 5, 1995, WO 95/18799, published Jul. 13, 1995 and U.S. Pat. No. 5,474,995, issued Dec. 12, 1995. Given the teachings of U.S. Pat. No. 5,543,297, entitled: “Human cyclooxygenase-2 cDNA and assays for evaluating cyclooxygenase-2 activity,” a person of ordinary skill in the art would be able to determine whether an agent is a selective COX-2 inhibitor or a precursor of a COX-2 inhibitor, and therefore part of the present invention.

An angiotensin system inhibitor is an agent that interferes with the function, synthesis or catabolism of angiotensin II. These agents include, but are not limited to, angiotensin-converting enzyme (ACE) inhibitors, angiotensin II antagonists, angiotensin II receptor antagonists, agents that activate the catabolism of angiotensin II, and agents that prevent the synthesis of angiotensin I from which angiotensin II is ultimately derived. The renin-angiotensin system is involved in the regulation of hemodynamics and water and electrolyte balance. Factors that lower blood volume, renal perfusion pressure, or the concentration of Na⁺ in plasma tend to activate the system, while factors that increase these parameters tend to suppress its function.

Angiotensin II antagonists are compounds which interfere with the activity of angiotensin II by binding to angiotensin II receptors and interfering with its activity. Angiotensin II antagonists are well known and include peptide compounds and non-peptide compounds. Most angiotensin II antagonists are slightly modified congeners in which agonist activity is attenuated by replacement of phenylalanine in position 8 with some other amino acid; stability can be enhanced by other replacements that slow degeneration in vivo. Examples of angiotensin II antagonists include: peptidic compounds (e.g., saralasin, [(San¹)(Val⁵)(Ala⁸)]angiotensin-(1-8) octapeptide and related analogs); N-substituted imidazole-2-one (U.S. Pat. No. 5,087,634); imidazole acetate derivatives including 2-N-butyl-4-chloro-1-(2-chlorobenzile) imidazole-5-acetic acid (see Long et al., J. Pharmacol. Exp. Ther. 247 (1), 1-7 (1988)); 4,5,6,7-tetrahydro-1H-imidazo[4,5-c]pyridine-6-carboxylic acid and analog derivatives (U.S. Pat. No. 4,816,463); N2-tetrazole beta-glucuronide analogs (U.S. Pat. No. 5,085,992); substituted pyrroles, pyrazoles, and tryazoles (U.S. Pat. No. 5,081,127); phenol and heterocyclic derivatives such as 1,3-imidazoles (U.S. Pat. No. 5,073,566); imidazo-fused 7-member ring heterocycles (U.S. Pat. No. 5,064,825); peptides (e.g., U.S. Pat. No. 4,772,684); antibodies to angiotensin II (e.g., U.S. Pat. No. 4,302,386); and aralkyl imidazole compounds such as biphenyl-methyl substituted imidazoles (e.g., EP Number 253,310, Jan. 20, 1988); ES8891 (N-morpholinoacetyl-(-1-naphthyl)-L-alanyl-(4, thiazolyl)-L-alanyl (35, 45)-4-amino-3-hydroxy-5-cyclo-hexapentanoyl-N-hexylamide, Sankyo Company, Ltd., Tokyo, Japan); SKF108566 (E-alpha-2-[2-butyl-1-(carboxy phenyl) methyl]1H-imidazole-5-yl[methylane]-2-thiophenepropanoic acid, Smith Kline Beecham Pharmaceuticals, PA); Losartan (DUP753/MK954, DuPont Merck Pharmaceutical Company); Remikirin (RO42-5892, F. Hoffman LaRoche AG); A₂ agonists (Marion Merrill Dow) and certain non-peptide heterocycles (G.D. Searle and Company).

ACE inhibitors include amino acids and derivatives thereof, peptides, including di- and tri-peptides and antibodies to ACE which intervene in the renin-angiotensin system by inhibiting the activity of ACE thereby reducing or eliminating the formation of pressor substance angiotensin II. ACE inhibitors have been used medically to treat hypertension, congestive heart failure, myocardial infarction and renal disease. Classes of compounds known to be useful as ACE inhibitors include acylmercapto and mercaptoalkanoyl prolines such as captopril (U.S. Pat. No. 4,105,776) and zofenopril (U.S. Pat. No. 4,316,906), carboxyalkyl dipeptides such as enalapril (U.S. Pat. No. 4,374,829), lisinopril (U.S. Pat. No. 4,374,829), quinapril (U.S. Pat. No. 4,344,949), ramipril (U.S. Pat. No. 4,587,258), and perindopril (U.S. Pat. No. 4,508,729), carboxyalkyl dipeptide mimics such as cilazapril (U.S. Pat. No. 4,512,924) and benazapril (U.S. Pat. No. 4,410,520), phosphinylalkanoyl prolines such as fosinopril (U.S. Pat. No. 4,337,201) and trandolopril.

Renin inhibitors are compounds which interfere with the activity of renin. Renin inhibitors include amino acids and derivatives thereof, peptides and derivatives thereof, and antibodies to renin. Examples of renin inhibitors that are the subject of United States patents are as follows: urea derivatives of peptides (U.S. Pat. No. 5,116,835); amino acids connected by nonpeptide bonds (U.S. Pat. No. 5,114,937); di- and tri-peptide derivatives (U.S. Pat. No. 5,106,835); amino acids and derivatives thereof (U.S. Pat. Nos. 5,104,869 and 5,095,119); diol sulfonamides and sulfinyls (U.S. Pat. No. 5,098,924); modified peptides (U.S. Pat. No. 5,095,006); peptidyl beta-aminoacyl aminodiol carbamates (U.S. Pat. No. 5,089,471); pyrolimidazolones (U.S. Pat. No. 5,075,451); fluorine and chlorine statine or statone containing peptides (U.S. Pat. No. 5,066,643); peptidyl amino diols (U.S. Pat. Nos. 5,063,208 and 4,845,079); N-morpholino derivatives (U.S. Pat. No. 5,055,466); pepstatin derivatives (U.S. Pat. No. 4,980,283); N-heterocyclic alcohols (U.S. Pat. No. 4,885,292); monoclonal antibodies to renin (U.S. Pat. No. 4,780,401); and a variety of other peptides and analogs thereof (U.S. Pat. Nos. 5,071,837, 5,064,965, 5,063,207, 5,036,054, 5,036,053, 5,034,512, and 4,894,437).

HMG-CoA reductase inhibitors useful for co-administration with the agents of the invention include, but are not limited to, simvastatin (U.S. Pat. No. 4,444,784), lovastatin (U.S. Pat. No. 4,231,938), pravastatin sodium (U.S. Pat. No. 4,346,227), fluvastatin (U.S. Pat. No. 4,739,073), atorvastatin (U.S. Pat. No. 5,273,995), cerivastatin, and numerous others described in U.S. Pat. No. 5,622,985, U.S. Pat. No. 5,135,935, U.S. Pat. No. 5,356,896, U.S. Pat. No. 4,920,109, U.S. Pat. No. 5,286,895, U.S. Pat. No. 5,262,435, U.S. Pat. No. 5,260,332, U.S. Pat. No. 5,317,031, U.S. Pat. No. 5,283,256, U.S. Pat. No. 5,256,689, U.S. Pat. No. 5,182,298, U.S. Pat. No. 5,369,125, U.S. Pat. No. 5,302,604, U.S. Pat. No. 5,166,171, U.S. Pat. No. 5,202,327, U.S. Pat. No. 5,276,021, U.S. Pat. No. 5,196,440, U.S. Pat. No. 5,091,386, U.S. Pat. No. 5,091,378, U.S. Pat. No. 4,904,646, U.S. Pat. No. 5,385,932, U.S. Pat. No. 5,250,435, U.S. Pat. No. 5,132,312, U.S. Pat. No. 5,130,306, U.S. Pat. No. 5,116,870, U.S. Pat. No. 5,112,857, U.S. Pat. No. 5,102,911, U.S. Pat. No. 5,098,931, U.S. Pat. No. 5,081,136, U.S. Pat. No. 5,025,000, U.S. Pat. No. 5,021,453, U.S. Pat. No. 5,017,716, U.S. Pat. No. 5,001,144, U.S. Pat. No. 5,001,128, U.S. Pat. No. 4,997,837, U.S. Pat. No. 4,996,234, U.S. Pat. No. 4,994,494, U.S. Pat. No. 4,992,429, U.S. Pat. No. 4,970,231, U.S. Pat. No. 4,968,693, U.S. Pat. No. 4,963,538, U.S. Pat. No. 4,957,940, U.S. Pat. No. 4,950,675, U.S. Pat. No. 4,946,864, U.S. Pat. No. 4,946,860, U.S. Pat. No. 4,940,800, U.S. Pat. No. 4,940,727, U.S. Pat. No. 4,939,143, U.S. Pat. No. 4,929,620, U.S. Pat. No. 4,923,861, U.S. Pat. No. 4,906,657, U.S. Pat. No. 4,906,624 and U.S. Pat. No. 4,897,402, the disclosures of which patents are incorporated herein by reference.

It is to be understood that the invention contemplates the use of one or more of any of the foregoing agents in combination with the repeated RIC regiment of the invention.

### RIC

As used herein, a RIC regimen is at least one cycle of an induced transient ischemic event followed by a reperfusion event. Typically, these regimens are performed by restricting blood flow in a limb or a peripheral tissue of the subject and then removing the blood flow restriction and allowing blood to reperfuse the limb or tissue. A regimen may comprise a single cycle or multiple cycles, including 2, 3, 4, 5, or more cycles. In one important embodiment, a regiment comprises 4 cycles of ischemia and reperfusion.

The blood flow restriction typically takes the form of an applied pressure to the limb or tissue that is above systolic pressure (i.e., supra-systolic pressure). It may be about 5, about 10, about 15, about 20, about 25, about 30, about 35 or more mm Hg above (or greater than) systolic pressure. Since systolic pressure will differ between subjects, the absolute pressure needed to induce ischemia will vary between subjects. In other embodiments the pressure may be preset at, for example, 200 mmHg. In other embodiments, it may be preset at about 160, about 170, about 180, about 190, about 200, about 210, about 220, about 230, about 240, about 250 mm Hg or higher. The blood flow restriction may be accomplished using any method as the invention is not limited in this regard. Typically, it may be accomplished with an inflatable cuff, although a tourniquet system is also suitable. Further examples of automated devices for performing RIC are described below.

The induced ischemic event is transient. That is, it may have a duration of about 1, about 2, about 3, about 4, about 5, or more minutes. Similarly, the reperfusion event may have a duration of about 1, about 2, about 3, about 4, about 5, or more minutes. The Examples demonstrate the effect of 4 cycles of 5 minutes of ischemia followed by 5 minutes of reperfusion on physical performance.

If performed using a limb, the upper limb or lower limb may be used although in some instances the upper limb is preferred. In some instances, RIC is performed on two different sites on the body, in an overlapping or simultaneous manner.

RIC may be performed using any device provided it is capable of inducing transient ischemia and reperfusion, whether manually or automatically.

In one of its simplest forms, the method may be carried out using a sphygmomanometer (i.e., the instrument typically used to measure a subject's blood pressure). The cuff of the sphygmomanometer is placed about a subject's limb (e.g., an arm or leg) and is inflated to a pressure great enough to occlude blood flow through the limb (i.e., a pressure greater than the subject's systolic blood pressure). The cuff is maintained in the inflated state to prevent blood flow through the limb for a specified period of time, referred to herein as the ischemic duration. After the ischemic duration, pressure is released from the cuff to allow reperfusion of blood through the limb for a period of time that is referred herein as the reperfusion duration. The cuff is then re-inflated and the procedure is immediately repeated a number of times.

The method may similarly be carried out using a manual type tourniquet. Devices such as those described in published PCT application WO 83/00995 and in published US application 20060058717 may also be used.

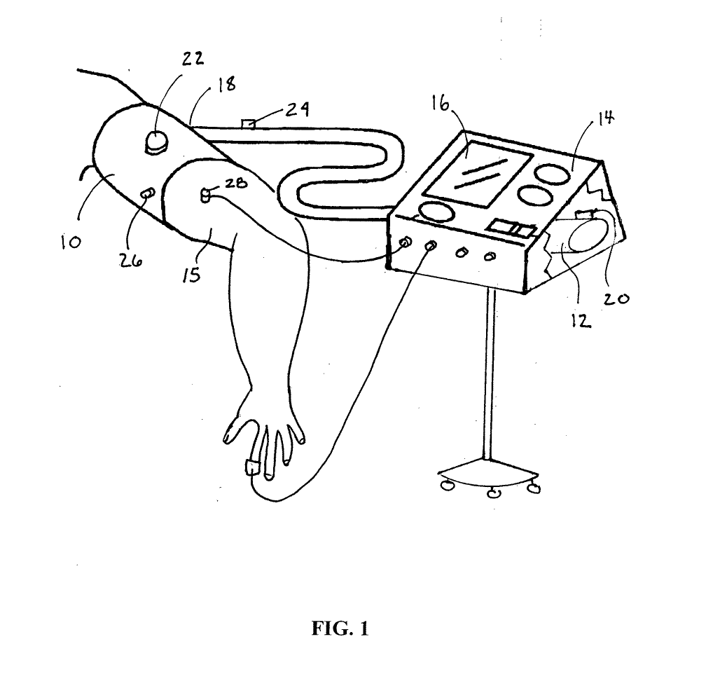

Another system that may be used is described in published US application 20080139949. The advantage of this system is that it can be used independently of a medical practitioner, and that it automatically induces the required RIC regimen. This system is exemplified in part in FIG. 1, which illustrates a cuff 10, an actuator 12, a controller 14 and a user interface 16. The cuff is configured to be placed about the limb 15 of a subject, such as an arm or leg of the subject. The actuator, when actuated, causes the cuff to retract about the limb to occlude blood flow through the limb. The controller executes a protocol that comprises repeating a cycle one or more times. The cycle itself includes actuating the cuff to prevent blood flow, maintaining the cuff in an actuated state for an ischemic duration, releasing the cuff, and maintaining the cuff in a relaxed state to allow reperfusion.

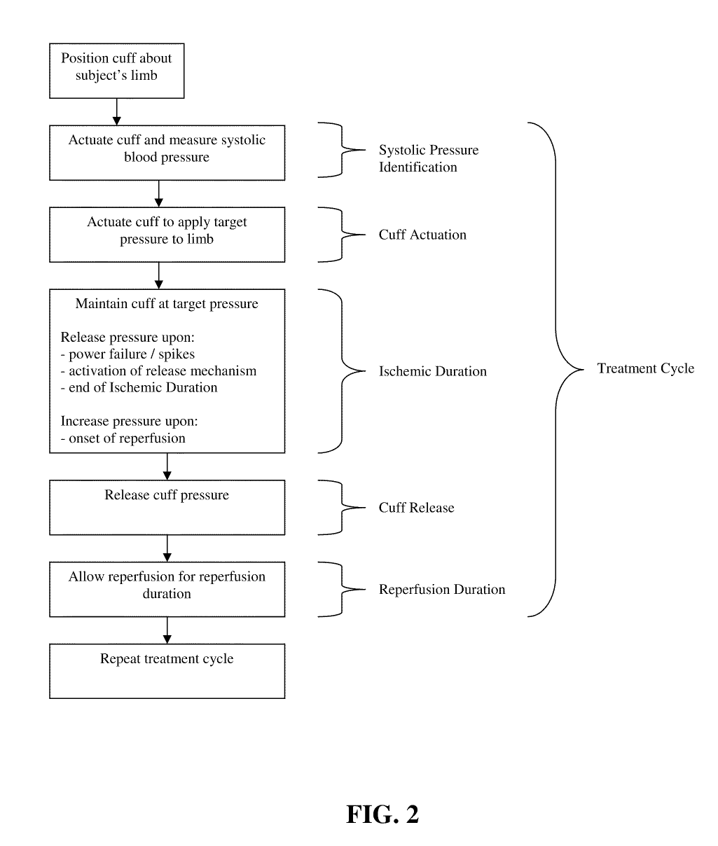

FIG. 2 shows a block diagram that represents a scheme that may be used to perform RIC. The scheme begins with placement of a cuff about a subject's limb. The system is then activated and the protocol is initiated through the controller. In one embodiment, the system is activated by a medical professional. In another embodiment, the system may be activated by the subject. The cuff contracts to apply an initial pressure, greater than systolic pressure, to the subject's limb. As discussed herein, the initial pressure may be a default value of the system or may be programmed into a particular protocol. The cuff then deflates to identify the subject's systolic pressure. This may be accompanied by monitoring the subject for the onset of Korotkoff sounds or vibrations. Alternatively or additionally, a distal remote sensor (e.g., a device on the fingertip which is sensitive to the presence or absence of flow or maintenance of flow) may be used. Once systolic pressure has been identified, the system initiates the first cycle of the protocol. In some embodiments, systolic pressure may be identified as an initial portion of the protocol. As used herein, the terms protocol and regimen are used interchangeably.

The cycle begins as the cuff contracts to apply a target pressure, greater than the subject's systolic pressure by an amount defined in the protocol, to the subject's limb. This occludes blood flow through the subject's limb. The external pressure against the subject's limb is held for an ischemic duration defined in the protocol. The system monitors the subject during the ischemic duration for pressure release criteria, which may include system power failure, system power spikes, and manual activation of quick release mechanism. The system also monitors the subject during the ischemic duration for any signs of reperfusion through the subject's limb, and accordingly, increases the external pressure applied by the cuff to prevent such reperfusion. Signs of reperfusion can include the onset of Korotkoff sounds or vibrations. After passage of the ischemic duration, the cuff releases pressure from about the subject's limb to allow reperfusion. Reperfusion is allowed for a reperfusion duration defined in the cycle.

The initial cycle typically concludes after the reperfusion duration. At this time, a subsequent cycle may begin as the cuff is actuated to contract about the subject's limb to occlude blood flow through the limb for another ischemic duration.

The cuff illustrated in FIG. 1 is configured to be positioned about the limb of a subject and to contract about the limb when actuated. In one embodiment, the sleeve is wrapped about a subject's upper arm, calf, or thigh and is fastened snuggly in place. Portions of the cuff may include hook and loop type material that can be used to fasten the sleeve in place about the subject's limb. The actuator inflates the cuff such that the limb is constricted to the point of occluding blood flow through the subject's limb.

The illustrated cuff includes an inflatable bladder (not shown) that receives a fluid, such as air, to cause the cuff expand and retract about a subject's limb. The bladder is constructed of an air impermeable material, such as flexible plastic or rubber. A connection port 18 is present at one end of the bladder to allow air to enter the bladder during inflation, or to exit the bladder during deflation. The port may include engagement features to facilitate a connection to the actuator, such as by an air hose. These features may include threads, clips, and the like. Although the illustrated embodiment includes a single bladder positioned within a cuff, it is to be appreciated that other embodiments are also possible. By way of example, according to some embodiments, the fabric sleeve may itself be air impermeable, such that no separate bladder is required. In other embodiments, multiple, separate inflatable bladders may be incorporated into a common sleeve, as aspects of the present invention are not limited in this respect.

The general size of subjects that undergo RIC may vary greatly, particularly given the range of species to which the methods may be applied. Given this variance, it may be desirable for some embodiments of cuffs to be adjustable over a wide range to accommodate the variety of subject limb girths that may be expected. According to some embodiments, the cuff comprises an inflatable fabric sleeve having a length greater than three feet, such that a girth of up to three feet may be accommodated. Embodiments of cuffs may include a width as small as two inches, one inch, or even smaller, so as to accommodate the upper arm or leg of a much smaller subject, including a neonatal infant. It is to be appreciated, however, that other embodiments may be configured to encircle a much smaller range of limb sizes, as aspects of the present invention are not limited in this regard.

Various devices may be used as an actuator to constrict the cuff about a subject's limb, or to release the cuff. As illustrated in embodiment of FIG. 1, the actuator includes a pneumatic pump to provide pressurized air to an inflatable cuff through an air hose. The actuator also includes a release valve 20 that, when actuated, opens a passageway between the inflatable cuff and the external environment to allow pressurized air to escape from the cuff, so that the cuff loosens about the subject's limb.

The air pump can comprise any device capable of delivering compressed air. According to some embodiments, the air pump includes a piston compressor, although other types of pumps, like centrifugal pumps and scroll compressor may also be used. The pump may be configured to provide air flow at a rate of between 0.1 to 20 cubic feet per minute, with a head pressure of up to 50 psi, according to some embodiments. However, other flow rates and/or pressures are possible, as aspects of the invention are not limited in this respect.

As discussed above, the actuator may also include a release mechanism to release a cuff from about the subject's limb. In the illustrated embodiment, the release comprises a release valve 20 that is positioned within the controller housing. The release valve, as shown, may be a solenoid that moves rapidly between fully closed and fully open positions to rapidly release air from the cuff and, in turn, to rapidly release the cuff from a subject. According to some embodiments, the same release valve or another release valve may also be actuated to open slowly, such as to adjust the pressure of the cuff or to allow a more controlled release of pressure such as may be required when the subject's blood pressure is measured.

Embodiments of the system may include safety features to allow rapid release of the cuff from a subject's limb. Moreover, some of these embodiments may be readily activated by a subject, such as when the subject feels discomfort. In one embodiment, the safety release 22 includes a large button positioned on or near the cuff. In this regard, the safety release is within reach of the subject. In other embodiments, the safety release may comprise a separate actuator, such as one that may be held in the free hand of the subject. Activating the safety release may cause the release valve of a pneumatic cuff to open, thereby allowing rapid removal of air from the cuff.

The system may also include a continually operating, cuff release mechanism. By way of example, a slow release valve may be incorporated into a pneumatic cuff to provide for a continual, slow release of pressurized air from the cuff. The continual slow release mechanism may provide for the safe release of a subject's limb, even in the face of power failures or other events that may prevent redundant safety features from operating properly. Similar type mechanism may be incorporated into embodiments that do not utilize a pneumatically inflatable cuff, as continual slow release mechanisms are not limited to pneumatic cuffs.

Embodiments of the system include a controller that receives information from a protocol and any other sensors in the system to, in turn, control the actuator to perform RIC. The controller and protocol combination may be implemented in any of numerous ways. For example, in one embodiment the controller and protocol combination may be implemented using hardware, software or a combination thereof. When implemented in software, the software code can be executed on any suitable processor or collection of processors, whether provided in a single computer or distributed among multiple computers. It should be appreciated that any component or collection of components that perform the functions described herein can be generically considered as one or more controllers that control the functions discussed herein. The one or more controllers can be implemented in numerous ways, such as with dedicated hardware, or with general purpose hardware (e.g., one or more processors) that is programmed using microcode or software to perform the functions recited above. The one or more controllers may be included in one or more host computers, one or more storage systems, or any other type of computer that may include one or more storage devices coupled to the one or more controllers. In one embodiment, the controller includes a communication link to communicate wirelessly, or via electrical or optical cable, to a remote location.

In this respect, it should be appreciated that one implementation of the embodiments of the present invention comprises at least one computer-readable medium (e.g., a computer memory, a floppy disk, a compact disk, a tape, etc.) encoded with a protocol in the form of a computer program (i.e., a plurality of instructions), which, when executed by the controller, performs the herein-discussed functions of the embodiments of the present invention. The computer-readable medium can be transportable such that the protocol stored thereon can be loaded onto any computer system resource to implement the aspects of the present invention discussed herein. In addition, it should be appreciated that the reference to a protocol or controller which, when executed, performs the herein-discussed functions, is not limited to an application program running on a host computer. Rather, the term protocol is used herein in a generic sense to reference any type of computer code (e.g., software or microcode) that can be employed to program a processor to implement the herein-discussed aspects of the present invention.

The system may also comprise one or more sensors 26 that receive information from the subject and/or portions of the system itself. Such sensors may receive information regarding blood flow in any portion of the subject, including the limb that is being treated. These sensors may also receive information regarding other operating parameters of the system, such as air pressure within a pneumatic cuff, direct readings of pressure applied by cuff, or tension within portions of a tension band.

Pneumatic cuffs may include a sensor to measure pressure within the cuff. Cuff pressure is often directly indicative of the pressure that exists within a blood vessel of the limb beneath the cuff. The controller of a system is often programmed to target a particular cuff pressure that is to be maintained during the ischemic duration of a cycle, as is discussed herein. In embodiments that include a pneumatic cuff, the pressure sensor may be positioned anywhere within the pressurized space of the cuff, the air hose, or even within the actuator itself. Pressure sensors may also be positioned on an inner surface of the cuff to directly measure the pressure between the cuff and an outer surface of the subject's limb. In use, the cuff may be oriented such that the pressure sensor is positioned directly above the subject's artery, so as to provide a more direct measurement of pressure at a blood vessel of interest.

In one embodiment, systems may also include one or more vibration and/or ultrasonic sensors 28 to identify Korotkoff sounds. Korotkoff sounds are generally understood to be present when pressures between systolic and diastolic are externally applied to the artery of a subject. Systolic pressure is associated with a pressure value that completely occludes blood flow through a subject's blood vessels, and in this regard, may be used by the system as feedback to identify when pressure in the system is low enough to allow blood flow, or high enough to occlude blood flow.

One or more sensors may be included to confirm the cessation of blood flow or reperfusion in the limb that receives the cuff. For instance, in some embodiments, a pulse oximeter 30 may be positioned on a distal portion of the limb that receives the cuff, such as on a finger or toe of the limb. The pulse oximeter can provide information regarding blood pulsing through the subject's blood vessels and the percentage of haemoglobin that is saturated with oxygen. The pulse oximeter will detect an absence of pulses when blood flow though a limb is not occurring to confirm the occlusion of blood flow. Moreover, the pulse oximeter may also detect the percentage of haemoglobin saturated with oxygen, which will drop as blood flow through the limb ceases. It is to be appreciated that other sensors may also be used to confirm the cessation of blood flow, such as a photoplethysmographic transducer, an ultrasonic flow transducer, a temperature transducer, an infrared detector, and a near infrared transducer, as aspects of the invention are not limited in this respect.

As mentioned above, the system includes a protocol that, through the controller, directs the operation of the system. Embodiments of the protocol include a cycle that comprises cuff actuation, an ischemic duration, cuff release, and a reperfusion duration. In many embodiments of protocols, the cycle may be repeated multiple times. Additionally, some embodiments of the protocol include systolic pressure identification.

The cuff actuation portion of the cycle comprises contracting the cuff about the limb of a subject to occlude blood flow through the limb. Contraction of the cuff is accomplished by the controller reading instructions from the protocol, such as a target set point for cuff pressure, and then by the initiating the controller to bring the cuff to the target set point. Attainment of the target set point may be sensed through any of the herein described sensors and techniques.

During the ischemic phase of the cycle, pressure is maintained about the subject's limb to prevent reperfusion of blood flow through the limb. The length of the ischemic phase, termed the ischemic duration, is typically defined by a doctor, or other medical professional, and is programmed into the protocol. Ischemic duration may be as short as a few seconds, or as long as 20 minutes, or even longer, as aspects of the invention are not limited in this regard. In some embodiments, the ischemic duration varies from cycle to cycle during the same protocol, although in other embodiments, the ischemic duration remains constant.

The controller acts to maintain pressure, applied by the cuff, at a set point above the subject's systolic pressure. Embodiments of the cuff may relax relative to the subject's limb over time, thereby reducing pressure and eventually allowing reperfusion. This may be caused by various factors, including relaxation of muscles in the subject's limb, stretching of the cuff about the limb, air leaks (intentional or unintentional), and the like. To this end, a sensor may provide pressure readings as feedback to the controller. The controller can measure any difference between the set point and the actual pressure reading and can provide any necessary commands to the actuator to compensate for errors.

Various approaches may be used to define an appropriate set point for the controller during the ischemic duration. According to one embodiment, the set point is manually entered into the protocol by the doctor (or other medical professional). Alternately, the doctor may select a set point in terms of the subject's systolic blood pressure. In one embodiment, the set point may be selected as a fixed pressure amount over the subject's systolic blood pressure, such as 5 mm Hg, 10 mm Hg, 15 mm Hg, 20 mm Hg, 25 mm Hg, 30 mm Hg, or any other fixed amount above systolic pressure of the subject. In other embodiments, the set point may be defined as a percentage of the subject's systolic blood pressure, such as 102% of systolic, 105%, 110%, 115%, and other percentages, as aspects of the invention are not limited in this respect. The point above systolic pressure may be set by the medical professional and may be dependent upon several factors including, but not limited to the size of the subject, the size of the subject's limb, the subject's blood pressure, confirmation of blood flow cessation, and the like.

The protocol, according to some embodiments, includes phases to identify the subject's systolic blood pressure. The cuff may be allowed to loosen about the subject's limb, from a point believed to be above systolic pressure, in a systematic manner while sensors are monitoring the limb for the onset of Korotkoff sounds or vibrations. Once the systolic pressure is identified, the protocol may continue in the normal course.

Identification of systolic pressure may optionally occur at any time during a protocol, or not at all. According to some embodiments, each cycle begins with the identification of the subject's systolic blood pressure. In other embodiments, systolic pressure may be identified only once during an initial portion of the protocol. In still other embodiments, systolic pressure may be identified as the cuff is released during the cuff release portion of each cycle. Still, as discuss herein, systolic pressure may not be identified at all during a protocol, as aspects of the invention are not limited in this regard.

The system can be configured to adjust the pressure set point during the ischemic duration. As discussed herein, the system may include sensors that detect the onset of reperfusion. As an example, this may be accomplished by detecting the presence of Korotkoff sounds or vibrations. The presence of Korotkoff sounds during an ischemic duration can indicate that either cuff pressure has fallen below systolic or that systolic pressure has risen above the set point that was previously above systolic pressure. Other devices may additionally or alternatively be used including for example devices on digits that detect the presence or absence of flow. In such a situation, the controller may adjust the set point based on the newly identified systolic pressure and/or other information and in this regard, can identify and prevent unwanted reperfusion that might otherwise occur.

The cuff release portion of a cycle occurs at the end of the ischemic duration and includes release of the cuff to a point below diastolic pressure. According to some embodiments, cuff release comprises releasing the pressure or tension of the cuff. In embodiments that utilize a pneumatic cuff, this may simply be associated with moving an air release valve to the fully open position to allow a rapid reduction in cuff pressure and a corresponding rapid relaxation of the cuff about the subject's limb. However, it is to be appreciated, that in other embodiments, that cuff relaxation may occur in a slower, more controlled manner, as aspects of the invention are not limited in this respect. Additionally, as discussed herein, the cuff release may be accompanied by monitoring for the onset of Korotkoff sounds or vibrations to identify or confirm the systolic pressure of the subject.

The reperfusion duration follows the cuff release in embodiments of the cycle. Reperfusion through the limb is allowed for a period of time termed the reperfusion duration. Much like the ischemic duration, reperfusion may be allowed for varied lengths of time, as short as a five seconds, one minute or more, and as long as 20 minutes, or even longer. The reperfusion duration may remain constant from cycle to cycle during a common protocol, or may vary between each cycle, as aspects of the invention are not limited in this respect.

The protocol may comprise any number of cycles. As discussed herein, a common cycle may simply be repeated a plurality of times, such as two, three, four, or more times, to complete a protocol. Alternately, the cycles of a protocol may be programmed with different parameters, such as different ischemic durations, reperfusion durations, pressure set points during the ischemic duration, and the like.

In some embodiments, the system may include a data logging feature that records the system parameters, such as cuff pressure or tension, during all phases of a protocol. Date of time of operation may also be recorded. Other features, such as personal information to identify the subject, may also be recorded by the system.

Embodiments of the system may incorporate various features to inform the subject or medical professional about the progress of the protocol. Audible or visual indicators may accompany any of the phases of the protocol. By way of example, a clock may show either the amount of time that has elapsed or that remains for a given portion of the protocol or the entire protocol. Embodiments may also include other features to keep the subject and/or medical professional informed, as aspects of the invention are not limited in this regard.

According to some embodiments, the system includes features to prevent tampering or accidental reprogramming by a subject. By way of example, in some embodiments, the reprogrammable features may only be accessed after entering a code. This can prevent a subject from mistakenly reprogramming the protocol or otherwise interfering with the operation of the system. It is to be appreciated that other devices may also be used to prevent accidental reprogramming, such as electronic keys, mechanical locks and the like.

The system may be configured for use is a variety of environments. By way of example, the system may be mounted on a portable stand with casters to facilitate easy movement. The stand may position the controller, user interface, and connections to the cuff at a convenient height for the subject. In other embodiments, the system is configured for portable use. In such embodiments, the system may be configured for ready placement into a suitcase for easy transport.

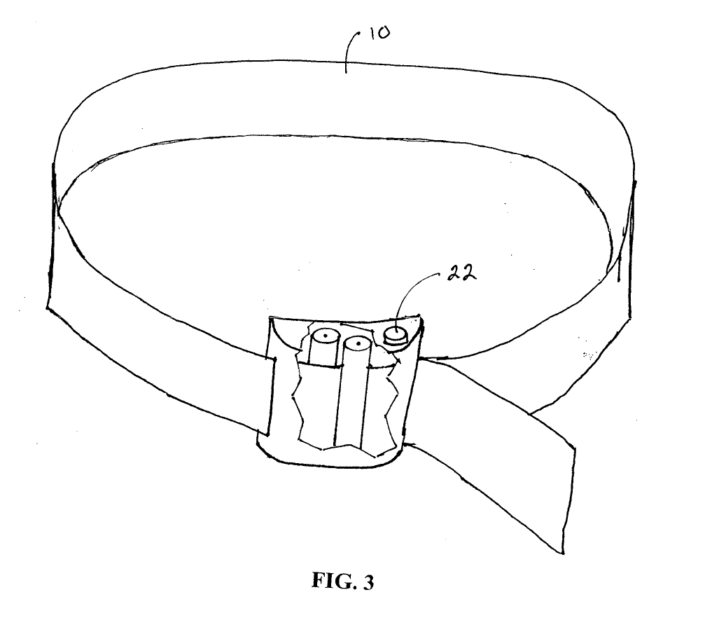

The system is also not limited to components illustrated in the embodiment of FIG. 1. By way of example, according to other embodiments, like that illustrated in FIG. 3, cuffs may be configured to constrict a subject's limb through alternative mechanisms. In the illustrated embodiment, the cuff is configured as a band having a ratcheting mechanism positioned at one end. In use, the band is wrapped about the limb of a subject with the free end of the band passing through the ratcheting mechanism. In such an embodiment, the actuator may comprise a mechanism that pulls the free end of the band further through the ratcheting mechanism to retract the cuff about the limb, or that frees the ratcheting mechanism to release the band to, in turn, release the band from the limb. Still other mechanisms, such as tourniquet mechanisms, are possible, as aspects of the invention are not limited in this respect.

As described above with reference to FIG. 3, some embodiments may have a cuff that comprises a band that does not inflate, but rather is tightened about a subject's limb by another mechanism. In such embodiments, the actuator may comprise a tensioning mechanism configured to move one end of the band relative to other portions of the band so as to place the band in tension. As shown, the mechanism can include opposed rollers held in close proximity to one another within a housing. The housing includes a slot for receiving a free end of the band and a fixation point for fixed attachment to the opposite end of the band. The free end of the band is passed into the slot and between the rollers. The rollers may be mechanically actuated to rotate relative to one another, such as by an electric motor, to pull the free end through the housing and thus tighten the band around a subject's limb.

The tensioning mechanism may include opposed rollers mounted on a ratcheting, free wheel mechanism. The freewheel mechanism allows the band to be pulled through the slot in one direction with minimal resistance so that the band may be pulled rapidly to a snug position about a subject's limb. The free wheel mechanism also prevents the band from moving through the slot in the loosening direction, unless the mechanism is released or the opposed rollers are actuated. It is to be appreciated that not all embodiments will include a free wheel mechanism, as aspects of the invention are not limited in this regard.

The opposed rollers rotate in either direction to tighten and loosen the band during use. When required, the rollers may rapidly rotate until the band achieves a particular tension. The rollers may further be actuated to make minor adjustments to the tension in the band during use. When the cuff is to be released from the subject's limb, a ratcheting mechanism or clutch may be released such that the opposed rollers are allowed to move freely, thus rapidly releasing tension.

The invention further contemplates kits that comprise devices for performing a medical intervention, such as for example a stent or a catheter, and components or whole devices for performing remote ischemic conditioning, such as for example a cuff such as a disposable cuff or a covering (e.g., sleeves) for the cuff that allows repeated use of the cuff without contamination. As an example, the invention contemplates a kit comprising a stent or catheter and a disposable cuff or a disposable liner or sleeve for a blood pressure cuff. The kit may comprise more than one cuff including two, three, four or more cuffs. The kit may comprise more than one liner or sleeve including two, three, four or more liners or sleeves.

Aspects of the invention are not limited to the embodiments of cuffs illustrated herein.

### EXAMPLES

### Methods

12 New Zealand White Rabbits (average weight 3.4 kilos) underwent intramuscular pentobarbital followed by nitrous oxide general anaesthesia. After endotracheal intubation, ventilation was mechanically controlled to maintain a PaCO₂ of 32-35 mmHg by intermittent blood gas analysis. After completion of instrumentation, the animals were stabilized for 5 minutes and then either a sham or real ischemic pre-conditioning stimulus was performed. The ischemic pre-conditioning stimulus consisted of 4 cycles of 5 minutes of left hind limb ischemia followed by 5 minutes of reperfusion. The tourniquet effectiveness was verified with pulse oximetry to the ipsilateral foot and visual inspection of pallor during ischemia and flushing upon reperfusion. The animals in the sham group were treated identically, with a similar duration of anaesthesia during which sham tightening of a tourniquet placed around the hind limb was performed, but with no constriction of blood flow or return during the sham ischemic period.

After the ischemic pre-conditioning or sham procedure, the surgeon, blinded to the randomisation, performed a right carotid artery cut down in all animals. Under direct visualization, a 3 French arterial sheath was placed in the right carotid artery and a 3 French judkins coronary guiding catheter was advanced over wire to the distal abdominal aorta where it bifurcates into the left and right iliac arteries. This was visualized by direct fluoroscopy using a c-arm camera present in the operating suite. Using radio contrast dye injections, a 0.14″ guide wire was directed down the right iliac artery and a 3 or 3.5 mm non-compliant balloon was placed over the wire in the midpoint of the right ileac artery. Attempts were made to place this approximately 20 mm from the distal aorta in an area of no side branches, and of linear caliber. The balloon was inflated to approximately 1.5 times the size of the iliac artery with 3 repetitive inflation cycles of 30 seconds inflation and 30 seconds deflation. Following balloon injury and after final angiography to ensure some antigrade flow, the balloon and guiding catheter were removed along with the carotid sheath and the right carotid artery was tied off and the cut down site sutured.

### Chronic Conditioning Protocol

Those animals randomized to remote pre-conditioning prior to balloon injury were also treated with repeated conditioning daily for a further 6 days after the balloon injury. This was achieved using an identical protocol of 4 cycles of 5 minutes ischemia followed by 5 minutes of reperfusion to the left hind limb with assessments identical to those described above. The animals were not anaesethized however, undergoing the conditioning stimulus with simple restraint. Those in the sham group at the time of vascular injury continued in the sham group for the follow up period. Instead of receiving remote conditioning, they underwent similar periods of restraint on a daily basis for 7 days with placement but without tightening of a tourniquet around the left hind limb.

All animals were then followed for 30 days following the index surgical procedure. At that point they were anaesthetized with pentobarbital and euthanized. The right and left iliac arteries were harvested. They were fixed and stained for analysis by a blinded observer. A visual assessment and ranking of the degree of vascular injury was performed for each animal, and 6 slices through each area of injury were obtained for detailed morphometry. The assessments of the vascular histology were performed by observers blinded to the treatment randomisation.

### Results

The results of the detailed morphometry are given in FIG. 4. In the animals randomized to receive chronic conditioning, there was a highly significant (p=0.02) reduction in neointimal proliferation in the sections studied with no evidence of any medial changes. Consequently there was a significant (p=0.04) reduction in the ratio of intima to medial thickness.

### CONCLUSION

These data suggest a reduction in vascular injury associated with chronic conditioning, there being an approximate 50% reduction in measurable vascular injury in these animals.

### EQUIVALENTS

The foregoing written specification is considered to be sufficient to enable one ordinarily skilled in the art to practice the invention. The present invention is not to be limited in scope by examples provided, since the examples are intended as mere illustrations of one or more aspects of the invention. Other functionally equivalent embodiments are considered within the scope of the invention. Various modifications of the invention in addition to those shown and described herein will become apparent to those skilled in the art from the foregoing description. Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth or illustrated in the drawings. The invention is capable of other embodiments and of being practiced or of being carried out in various ways.

Also, the phraseology and terminology used herein is for the purpose of description and should not be regarded as limiting. The use of “including”, “comprising”, or “having”, “containing”, “involving”, and variations thereof herein, is meant to encompass the items listed thereafter and equivalents thereof as well as additional items.

What is claimed is:

1. A method for reducing restenosis in subject comprising
performing a repeated remote ischemic conditioning (RIC) regimen on a subject having or at risk of developing restenosis, in an amount and a frequency sufficient to reduce the incidence or severity of restenosis.

2. The method of claim 1, wherein restenosis occurs following a medical intervention.

3. The method of claim 1, wherein the repeated RIC regimen comprises more than one RIC regimen performed on a single day.

4. (canceled)

5. The method of claim 1, wherein the repeated RIC regimen comprises one or more RIC regimens on more than one day.

6. The method of claim 5, wherein the repeated RIC regimen comprises one or more RIC regimens performed on a daily basis for one month.

7. The method of claim 5, wherein the repeated RIC regimen comprises one or more RIC regimens performed every other day for one month.

8-9. (canceled)

10. The method of claim 1, wherein the subject is human.

11. The method of claim 1, wherein the subject will receive a medical intervention.

12. The method of claim 11, wherein the repeated RIC regimen is performed before the medical intervention.

13. The method of claim 11, wherein the repeated RIC regimen is performed during the medical intervention.

14. The method of claim 11, wherein the repeated RIC regimen is performed after the medical intervention.

15. The method of claim 11, wherein the repeated RIC regimen is performed before and after the medical intervention.

16. The method of claim 1, wherein the medical intervention is an intravascular stent placement.

17-20. (canceled)

21. The method of claim 11, wherein the medical intervention is angioplasty.

22. The method of claim 11, wherein the medical intervention is a non-vascular stent placement.

23. (canceled)

24. The method of claim 11, wherein at least one RIC regimen is performed within 24 hours of the medical intervention.

25-26. (canceled)

27. The method of claim 1, wherein at least one RIC regimen comprises at least four cycles, each cycle comprising supra-systolic pressure and reperfusion.

28. The method of claim 1, wherein at least one RIC regimen comprises more than one cycle comprising 5 minutes of supra-systolic pressure and 5 minutes of reperfusion.

29. The method of claim 27, wherein the supra-systolic pressure is a pressure that is at least 15 mmHg above systolic pressure.

30. The method of claim 1, wherein the repeated RIC regimen is performed at the same site.

31-34. (canceled)
